(19) United States
(12) Patent Application Publication
(10) Pub. No.: US 2007/0122844 A1
(43) Pub. Date: May 31, 2007

(54) REDUCTION OF REDUNDANT PROTEIN IDENTIFICATION IN HIGH THROUGHPUT PROTEOMICS

(75) Inventors: Robert E. Kearney, Montréal, QC (CA); John J. M. Bergeron, Pointe-Claire, QC (CA); Alexander Bell, Montréal, QC (CA); Peter McPherson, Lachine, QC (CA); Francois Blondeau, Montréal, QC (CA); Mathieu Drapeau, Montréal, QC (CA); Florence Servant, Pointe-Claire, QC (CA); Sebastien De Grandpre, Montréal, QC (CA); Annalyn Gilchrist, Somerville, MA (US); Souad Lesimple, Roxboro, QC (CA); Catherine Au, Montréal, QC (CA)

Correspondence Address: BERESKIN AND PARR, 40 KING STREET WEST, BOX 401, TORONTO, ON, M5H 3Y2, CA

(73) Assignee: MCGILL UNIVERSITY, Montreal (CA)

(21) Appl. No.: 11/470,166

(22) Filed: Sep. 5, 2006

Related U.S. Application Data
(60) Provisional application No. 60/713,373, filed on Sep. 2, 2005.

(51) Int. Cl. G01N 33/53; G06F 19/00

(52) U.S. Cl. 435/7.1; 702/19

(57) ABSTRACT

There is provided a method for the identification of proteins with reduced redundancy in protein hits. The method eliminates protein hits that are described by peptides sets that are included in at least one other protein hit associated peptides set.

### CROSS-REFERENCE TO RELATED APPLICATION

This application claims priority from U.S. provisional application No. 60/713,373 filed Sep. 2, 2005 and entitled METHOD FOR IDENTIFYING PROTEIN.

### FIELD OF THE INVENTION

The present invention relates to the field of proteomics. More specifically, the invention relates to the identification of proteins in a protein mixture using peptides and protein databases.

### BACKGROUND OF THE INVENTION

A fundamental goal of proteomics is the systematic simultaneous analysis of large numbers of proteins in biological samples. Automated, high-throughput analyses of complex protein mixtures are presently a matter of routine, made possible by the application of soft-ionization methods to mass spectrometry, and the sequencing of an ever increasing number of genomes. These innovations permit the identification and characterization of proteins with greater sensitivity, shorter analysis times, more consistency in the analysis process, and the flexibility of multiple assays. Global analyses such as these will provide a comprehensive framework within which more traditional, studies directed to individual proteins can be carried out.

In shotgun proteomics, protein samples are generally enzymatically digested into smaller peptide fragments to make them amenable to sequence analysis by mass spectrometry [1]. The resulting complex peptide sample is then separated in time, using liquid chromatography (LC), and coupled to a tandem mass spectrometer so that peptides can be detected and selected for fragmentation as they elute.

Tandem mass spectrometry uses two mass analyzers. The first mass analyzer selects a single peptide mass from the initial mass spectrum (MS) by filtering out all other masses. The single peptide is then fragmented in a collision cell and the second mass analyzer acquires the resulting fragmentation spectra (MS/MS). Peptides typically fragment along the polypeptide backbone rather than in the side chains. Consequently, the series of ions generated by fragmentation can be used to determine the amino acid sequence of the peptide. Protein database searches find all candidate peptides that match the mass of the parent ion to peptides in silico protein digests, then rank the candidates based on the matching theoretical and experimental fragmentation spectra [2, 3]. Proteins containing the identified peptides are then considered to have been identified. There is growing evidence that the number of MS/MS mass spectra (queries) associated with a protein identification provide a measure of relative protein abundance [4, 5].

Unfortunately, identification of proteins in this way yields a redundant list of proteins due to redundancies in peptide identifications, redundant database entries, and gene products that have long stretches of conserved sequence identity. This redundancy must be eliminated to correctly interpret the biological significance of the results or to peptide counts to estimate abundance. A common approach is to group the protein hits on the basis of sequence similarity (e.g. [6]); this is laborious, time-consuming, subjective and is based on derived results (protein sequence) rather than primary data (peptide sequence). Another approach uses a probabilistic analysis to select the proteins with the highest likelihood of being present based on a knowledge of the probability that the individual peptide identifications are correct [7].

### SUMMARY OF THE INVENTION

The present invention provides a simpler, set-based approach to the elimination of redundant protein identifications that yields the minimum number of proteins needed to explain the peptides observed.

In a broad embodiment of the invention, there is provided a method for identifying proteins in a mixture of proteins comprising: providing peptides derived from the mixture of proteins; obtaining mass spectra of the peptides to identify the peptides by comparing the mass spectra with spectra of a standardized database; matching the identified peptides with proteins in a database to generate a protein hits (PHs) list, each of the PHs having an associated peptides set; and identifying PHs having an associated peptides set that is included in at least one other PH-associated peptide set; and removing the identified PHs from the list and wherein remaining PHs provides an identification of the one or more proteins.

In another embodiment there is provided method as described above further comprising grouping the identified PHs that share a same set of peptides in primary protein groups and wherein each of the primary protein group identifies a non-redundant PH.

In another aspect the method can also comprise combining all primary protein groups that share at least one common characteristic among the non-redundant PH to generate secondary protein groups and identifying a non-redundant PH for each of the secondary protein groups based on the characteristic.

In another embodiment there is provided a method for reducing redundancy in a protein hits list, comprising: associating a set of peptides with each protein of the protein hits to generate PHs-associated peptide sets; comparing the set PHs-associated peptide sets; identifying PHs having an associated peptides set that is included in at least one other PH-associated peptides set; and removing the identified PHs from the list and wherein remaining PHs provides an identification of the one or more proteins.

The invention also provides a device for identifying proteins in a mixture of proteins, the device comprising a data input means for inputting peptide analysis results, a peptide database, a protein database, a first analyzer to identify the peptides, a second analyzer to match the identified peptides with proteins in the protein database to create protein hits (PH) and to create peptide sets associated with PHs, a comparator for comparing PH associated sets of peptide and for eliminating redundancy in PHs, and a display to display identified PH substantially free of redundancy.

In another embodiment, the invention also provides a computer readable medium with computer executable instructions for performing a method for identifying proteins comprising matching identified peptides obtained from a protein mixture with proteins in a database to generate protein hits (PH) each of said PHs having an associated peptide set; and eliminating PHs having a peptide set that is included in at least one other PH-associated peptide set thereby producing a set of PHs substantially free of redundancy.

### BRIEF DESCRIPTION OF THE DRAWINGS

Further features and advantages of the present invention will become apparent from the following detailed description, taken in combination with the appended drawings, in which:

FIG. 1 is an example of information contained in a protein hits (PH) array;

FIG. 2 is a graphic showing proteins hits and their associated peptides for a hypothetical proteomics experiment demonstrating how peptides may be shared among hits in various ways;

FIG. 3 is a table array showing the correspondence between PHs and peptides sets from the data of FIG. 2;

FIG. 4 is a distribution of the number of proteins (from rat) containing peptides having 6-30 amino acids;

FIG. 5 is a table array showing the correspondence between primary protein groups, PHs and PEPTIDEID;

FIG. 6 is a flow chart algorithm to group PHs;

FIG. 7 is a schematic representation of a result of adjacency analysis showing the connectivity between secondary groups;

FIG. 8 is a graphic of PHs and associated peptides in a typical proteomics experiment;

FIG. 9 is a graphic showing the results of applying the method of the invention to the data of FIG. 8;

FIG. 10 is a graph showing the linkage for secondary grouping for the ABRF sample;

FIG. 11 is a schematic representation of the sequences of PHs in a secondary group from FIG. 10 wherein horizontal bars represent areas of common peptides and stars represent areas of different peptides;

FIG. 12 is a graph showing the relative abundance of the 8 proteins in the ABFR sample estimated from the redundant peptide counts compared to knowon values.

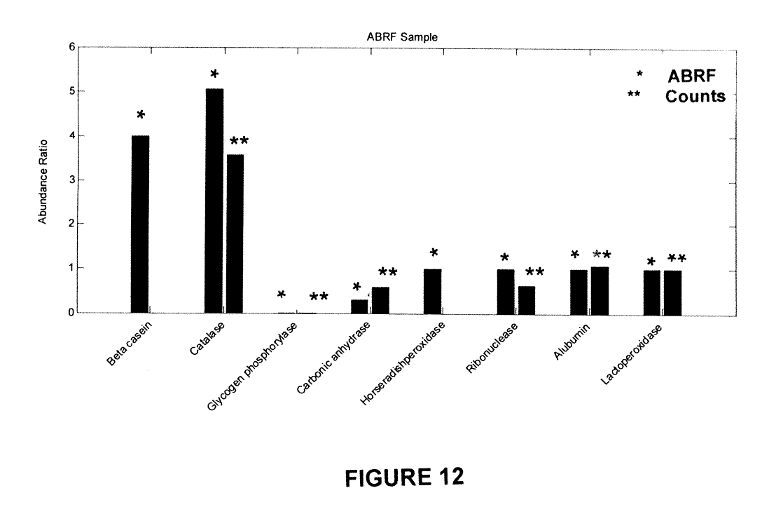

### DETAILED DESCRIPTION OF THE INVENTION

Protein Identification

A. Data Representation

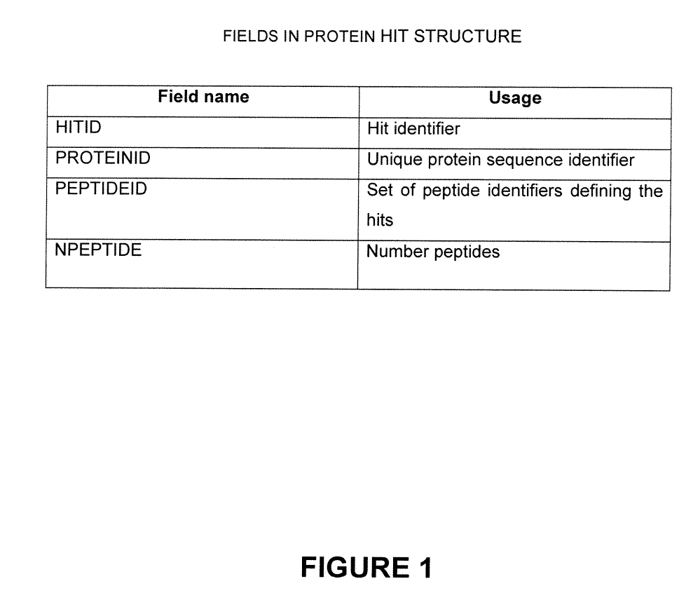

Protein identification algorithms operate in three stages. First, experimental fragmentation (ms/ms) mass spectra are matched to theoretical spectra from an in silico digestion of sequences in a protein database. Next, the matches are examined in some way to determine those which are valid. Finally, the proteins containing identified peptides are determined. Irrespective of the tools used, the results may be considered to consist of a set of protein hits PHs, each comprising a protein identifier and the associated set of peptides used to identify it. For example, let us assume that the protein hits are stored as a structure array, PH, having the fields defined in FIG. 1. It will be appreciated that the array can contain other information associated with a particular PH such as for example functional information regarding the identified protein, species (taxonomy) from which the protein sequence is derived, number of associated peptides and the like.

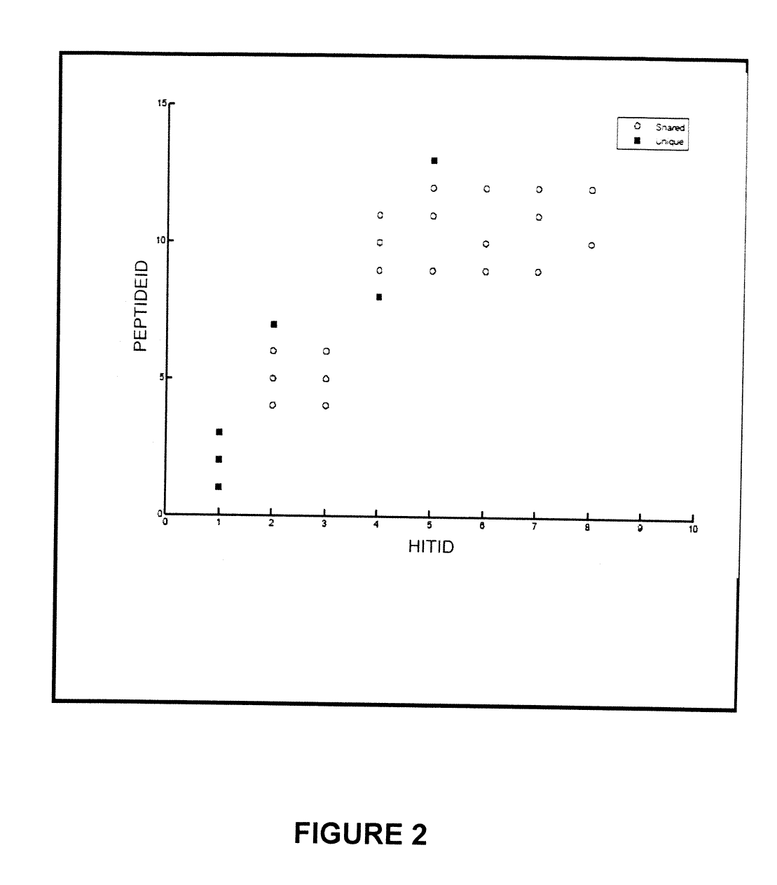

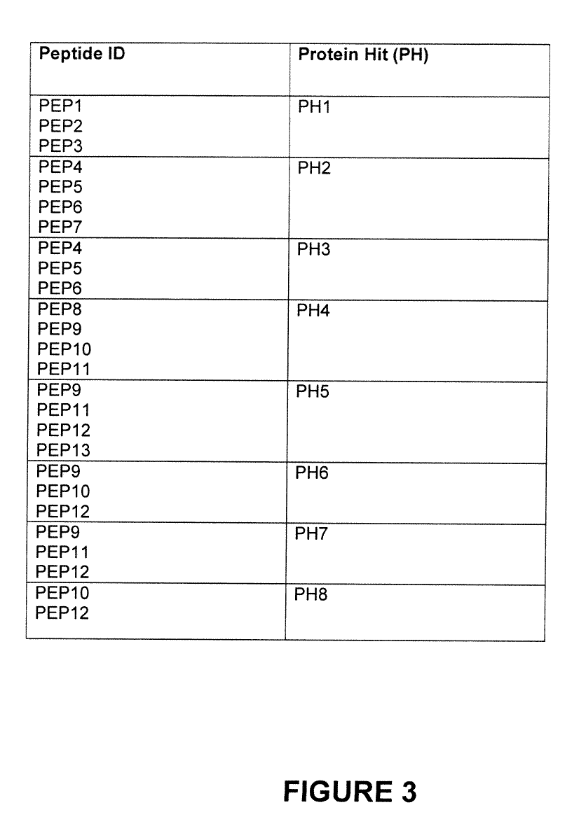

In practice, the protein hits resulting from the analysis of complex mixtures are found to be quite redundant. This is illustrated in FIG. 2 which shows the results of a hypothetical experiment in which 13 peptides were identified leading to the generation of 8 protein hits. However, inspection of this plot reveals that only 4 hits (1,2,4,5) have peptides which occur uniquely. Thus, the peptides for hit 3 are a subset of those for hit 2 while the peptides of hits 6, 7 and 8 are also found in hits 4 & 5. Indeed the peptides of hit 7 are a subject of hit 5 while the same applies to hits 8 and 6. The data of FIG. 2 are reproduced in tabular array in FIG. 3.

Moreover, there are cases where the peptides from one hit are a subset of those identifying another (e.g. hits 3 and 2 in FIG. 2). That is
[PH (i).PEPTIDEID]⊂[(PH). PEPTIDEID]

Such hits are redundant since postulating the existence of protein j can explain all of the peptides in both hits i and j. There is no evidence that protein i is present although its existence cannot be ruled out.

B. Redundant Peptide Identifications

The first source of redundant protein identifications is that a single mass spectrum may be matched to more than one peptide. Search algorithms, such as Mascot™ and Sequest™ [2, 3], identify peptides by matching fragmentation spectra to an in silico digest and evaluating the goodness of fit in some way. There are a number of amino acids whose masses cannot be distinguished by mass spectra data (e.g. isolucine and leucine are structural isomers while lysine and glutamine have the same nominal mass). Consequently, peptides whose sequences differ only by interchanges of such amino acids cannot be distinguished by mass spectra and so will result in redundant peptide identifications. In addition, there may also be cases in which an experimental spectrum matches more than one theoretical spectra well. Examination of a number of data sets from rat liver organelles revealed that approximately 5% of the mass spectra match two or more peptides.

C. Redundant Peptide to Protein Mapping

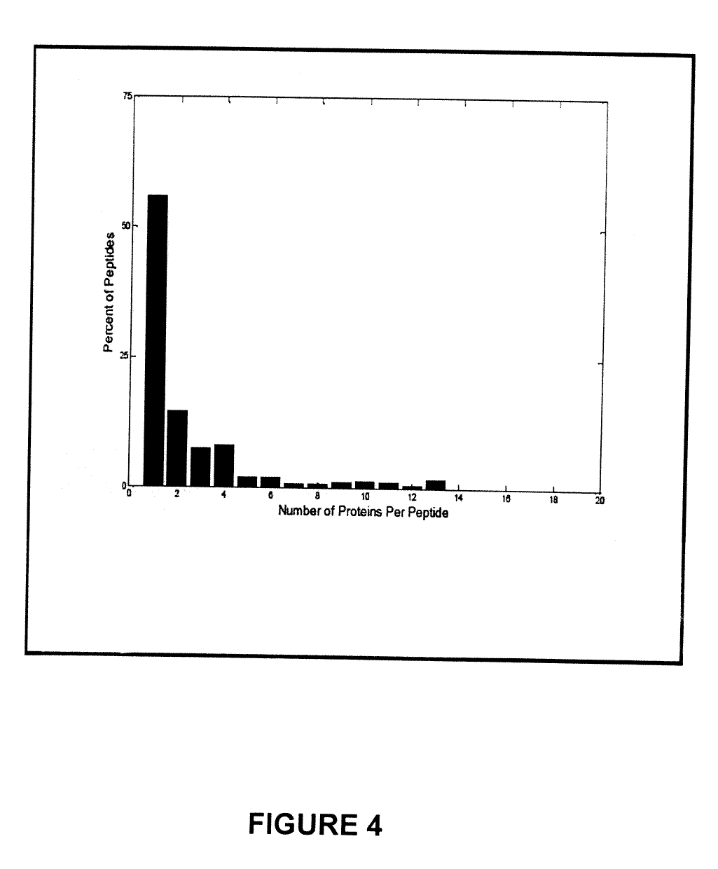

A second source of redundant protein identifications is that a particular peptide may occur in more than one protein sequence in the database. This can result from database inconsistencies including redundant entries in the database, partial sequences, and splice variants. It may also arise biologically from proteins that are closely related gene products having long stretches of conserved sequences as occurs in closely related gene products. An in silico analysis of all the tryptic peptides in the NCBI nr database [8] with taxonomy restricted to rat, suggests that only about 15% of peptides occur in more than one protein sequence. However, tandem mass spectrometry only identifies peptides between 6 and: 30 amino acids. These shorter peptides are much less specific and as FIG. 4 shows, more than 45% of these peptides occur in two or more proteins. The number of redundant peptides can be expected to increase when searches are carried using a wider range of taxonomies.

In the present invention there is provided a set-based algorithm that eliminates or reduces redundancy in protein identification. The method can be applied to already established list of PHs or may include the preparation of peptides using enzymatic digestion and mass spectrometry to identify the peptides and the proteins using standardized databases. In one embodiment all PHs that have a peptides set that is included in any other PH are eliminated from the PHs list. The remaining PHs provide an identification of the protein(s) in the mixture of proteins.

Protein hits, PHs, that share the same set of peptides can be grouped together to form a protein group PG. For a PG,
[PH (i).PEPTIDEID]⊂[PG.PEPTIDEID] ∀ᵢ in PG

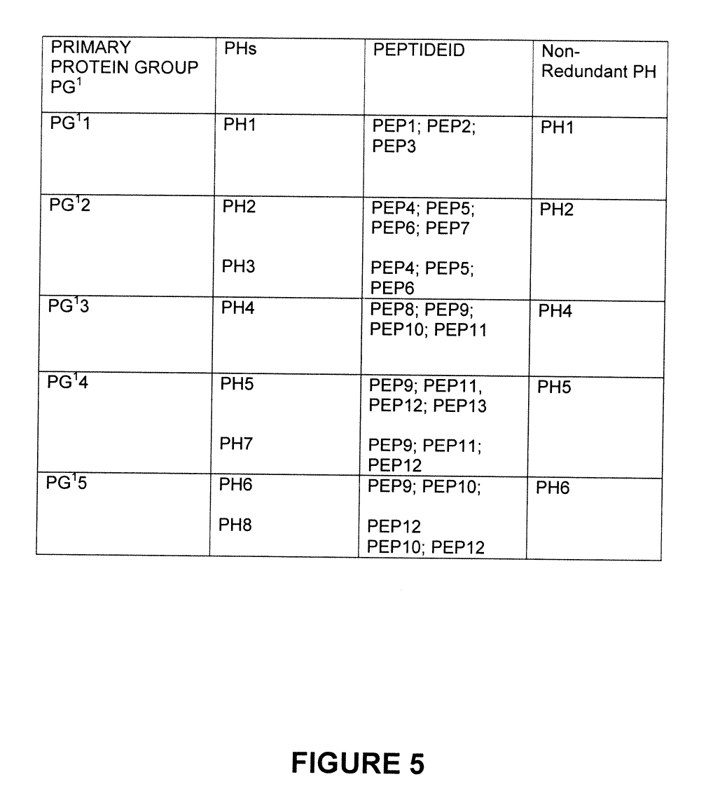

In the present description a group defined based on the above definition is referred to as a primary Protein Group or PG¹. FIG. 5 provides an example of PG¹s formed based on the above definition and on the data of FIGS. 2 and 3. PG¹2, PG¹4 and PG¹5 comprise more than one PH. Not all protein hits in a group need have all the peptides associated with the group. Within a group the protein comprising the most peptides (NPEPTIDEID) is identified as the nun-redundant PH (the other peptides being redundant) and is included in the protein list that serves to identify the proteins in a mixture. In other words the redundant PHs are eliminated from the protein list.

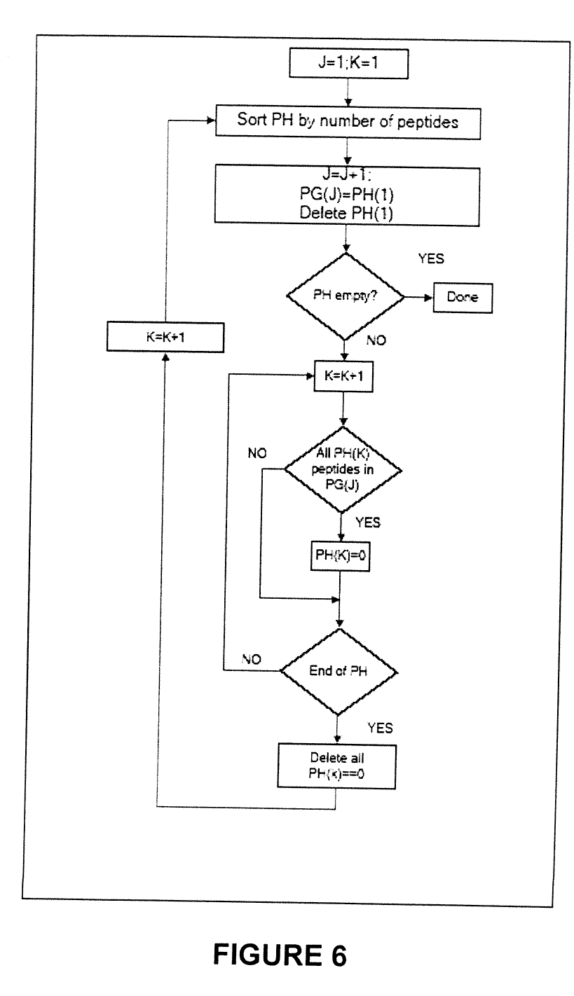

The algorithm used to define the protein identification group is illustrated in FIG. 6. It takes as its input PH, a structure array of redundant protein hits, and generates the output PG¹, a structure array containing the non redundant protein identification groups.

Groups can be defined iteratively by first sorting the protein hits by the number of peptides they each contain. Then all hits defined by sets of peptides contained within the initial set are found and merged into the first group. Hits assigned to a group are eliminated from the list of protein hits and the procedure repeated until all hits have been assigned.

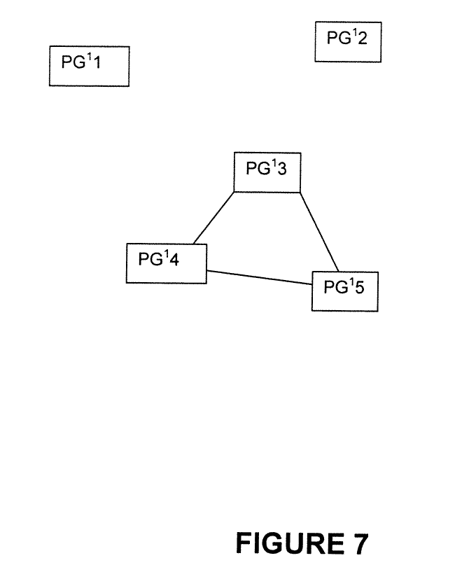

Redundancy can be further reduced by performing an adjacency analysis of the primary protein groups. This analysis joins primary protein groups that share at least one peptide among themselves into secondary protein groups. That is to primary protein groups for which the non-redundant PHs share at least one peptide are placed in a secondary protein group. Then the connectivity of each primary protein group within a secondary protein group is established. By connectivity it is meant the number of primary protein groups with which a given primary protein group shares at least one peptide. Referring back to FIG. 5, it can be seen that PG¹3, PG¹4 and PG¹5 share PEP9 and would therefore be grouped as a secondary protein group. It can further bee seen that the connectivity for PG¹3, PG¹4 and PG¹5 is 2. That is to say PG¹3 is connected with the other two groups (PG¹4 and PG¹5) and similarly for PG¹4 and PG¹5. Secondary grouping with connectivity is shown in FIG. 7.

The redundant PHs of a secondary protein group can be determined based on the connectivity. Thus for example, the primary protein group having the highest connectivity can be identified as the non-redundant PH of a secondary protein group. All other primary protein group associated non-redundant PHs would be eliminated from the list of PHs.

It will be appreciated that proteins that are identified as being redundant using the adjacency analysis are proteins for which the sequences are potentially highly related. For example a same protein obtained from different species, proteins exhibiting allelic variations, proteins in a database with sequencing errors and the like.

It will also be appreciated that criteria other than or in addition to peptide sharing among primary protein groups could also be applied in the adjacency analysis. For example, secondary grouping could be based on protein function, protein length and other such protein characteristics.

Query Counting

There is growing evidence that the number of MS/MS mass spectra (queries) associated with a protein identification are related in some way to the protein abundance [4, 5]. Consequently, the mass spectra information underlying the identification of each group is summarized by counting the associated peptides. Three peptide counts can be determined for each group. Thus,
N_{U} is the number of peptides which occur only in the group
N_{S} is the number of peptides that are shared with other groups
N_{P} is the pro-rated number of peptides that combines N_{U} with N_{S} weighted by the relative number of unique queries in the associated queries.
It is defined by:

$${N}_{P} ( i ) = {N}_{U} ( i ) + {N}_{S} ( i ) [ \frac{{N}_{U} ( i )}{{\sum }_{j = 1}^{j = nhits} {N}_{U} ( j )} ]$$

Thus the relative abundance of a non-redundant PH can be determined by providing a count of all the queries (peptides) associated with the corresponding primary or secondary protein group.

The method of the invention can be implemented in part using computer-based system and methods as would be known to one skilled in the art.

The invention also provides a device for identifying proteins in a mixture of proteins, the device comprising a data input means for inputting peptide analysis results, a peptide database, a protein database, a first analyzer to identify the peptides, a second analyzer to match the identified peptides with proteins in the protein database to create protein hits (PH) and to create peptide sets associated with PHs, a comparator for comparing PH associated sets of peptide and for eliminating redundancy in PHs, and a display to display identified PH substantially free of redundancy.

In another embodiment, the invention also provides a computer readable medium with computer executable instructions for performing a method for identifying proteins comprising matching identified peptides obtained from a protein mixture with proteins in a database to generate protein hits (PH) each of said PHs having an associated peptide set; and eliminating PHs having a peptide set that is included in at least one other PH-associated peptide set thereby producing a set of PHs substantially free of redundancy.

### EXAMPLES

### Example 1

We evaluated the algorithm by analyzing a representative data set from an organellar proteomics experiment using methods similar to those described in [4]. The raw data comprised 13,587 tandem mass spectra acquired from 93 bands from a 1 D gel of a sample of rat rough microsome. Mass spectra were first subjected to peak-detection using a commercial product (Mascot Distiller from Matrix Science) and the resulting peak-lists searched against the NCBI nr database [8] with taxonomy limited to rat using a probability-based search engine (Mascot from Matrix Science). A total of 5,685 mass spectra were assigned to peptides with a probability of random hit being less than 5%. There were 3,498 distinct peptide identifications. The search results were loaded into CellMapBase, our relational database for proteomics analysis [9] and analyzed using the method of the invention.

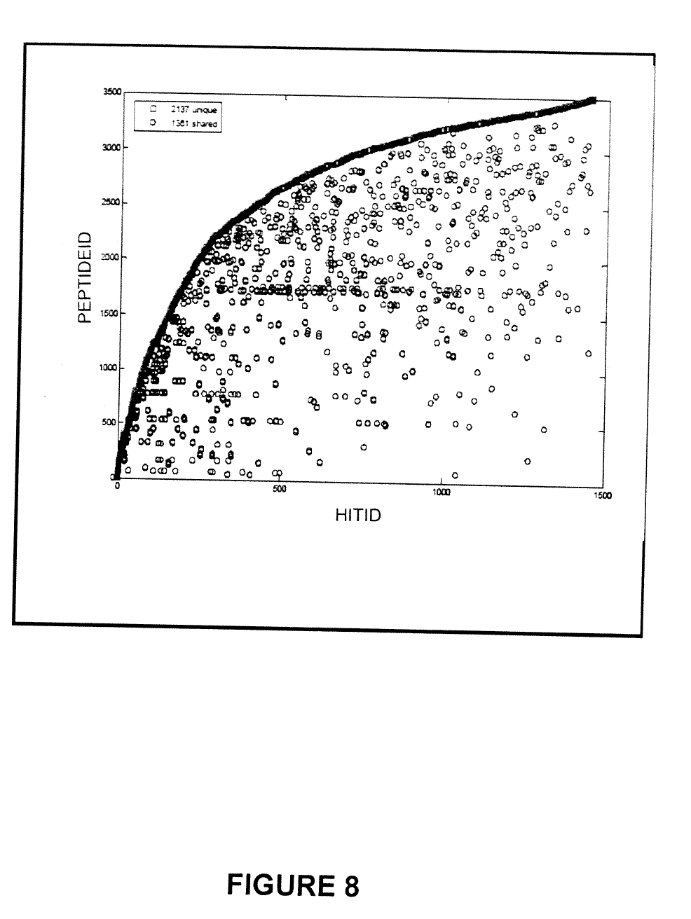

FIG. 8 illustrates the distribution of peptides across the protein hits identified from this data set. As in FIG. 2, it is evident that there are many shared peptides. Indeed more than a third of the protein hits contain one or more peptides that are shared among at least two hits. The complexity of this plot illustrates the difficulty of attempting to eliminate redundant identifications by manual analysis.

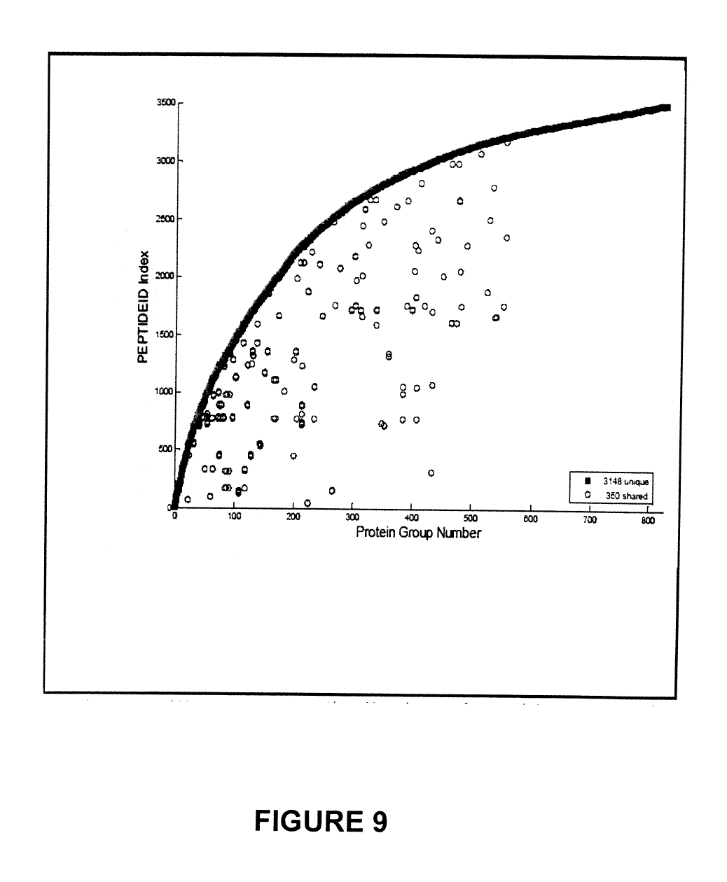

FIG. 9 shows the results of applying the grouping algorithm to the data from FIG. 8. It is evident that the number of proteins identified (protein groups) is substantially smaller and there are far fewer shared peptides.

Table II provides the quantitative support for this information. Grouping decreased the number of proteins identified by more than 40% and increased the number of proteins identified by unique peptides from 512 to 600. Taken together, the percentage of identifications using only unique peptides from 35.2% to 80.1%.

This grouping algorithm provides an objective, automated means to eliminate redundancy in protein identifications in high throughput proteomic experiments. However, as FIG. 9 demonstrates, it does not completely eliminate shared peptides, presumably reflecting the presence of distinct, but closely related proteins. The algorithm also identifies a few groups, (e.g. hits 6 & 8 in FIG. 2) with only shared peptides that cannot be assigned to any protein with confidence.

### Example 2

The Association of Biomolecular Resource Facilities (ABRF) recently circulated two samples containing 8 proteins in different amounts to assist laboratories in evaluating their ability to identify and quantify unknown proteins. This example describes the analysis of these samples using the proteomics pipeline.

Analysis Methods

The two ABRF samples were resolved on separate 1D-SDS PAGE gel lanes and subjected to standard band slicing, in-gel trypsinization and LC-coupled mass spectrometry. Peak lists were generated using. Mascot Distiller with optimized parameter values. Peptides were identified using Mascot to search the NCBI nr database with taxonomy limited to mammals. Peptides identified in the two samples were used to identify the proteins present and group them, according to the method described above into distinct sets to define the minimal set of proteins necessary to explain the observed peptides.

Table 2 shows the 59 protein groups defined by distinct sets of peptides initially identified.

Adjacency Analysis (Secondary Grouping)

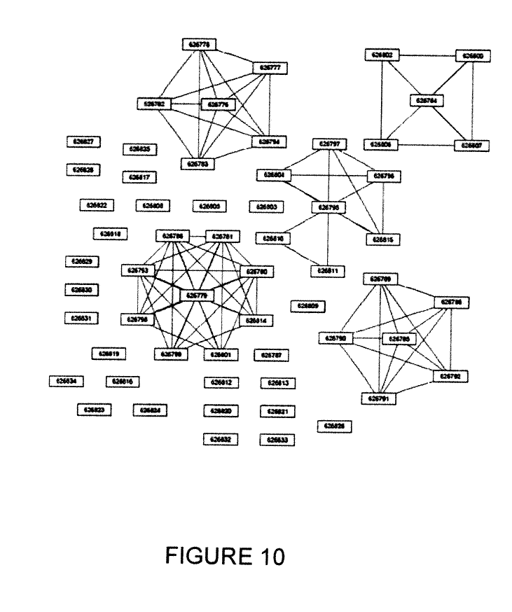

Sets of closely related proteins groups were determined by adjacency analysis to generate secondary protein groups. FIG. 10 shows a graph of the relations between groups. Five “islands”—sets of groups which share peptides only among themselves—are apparent

Related Proteins

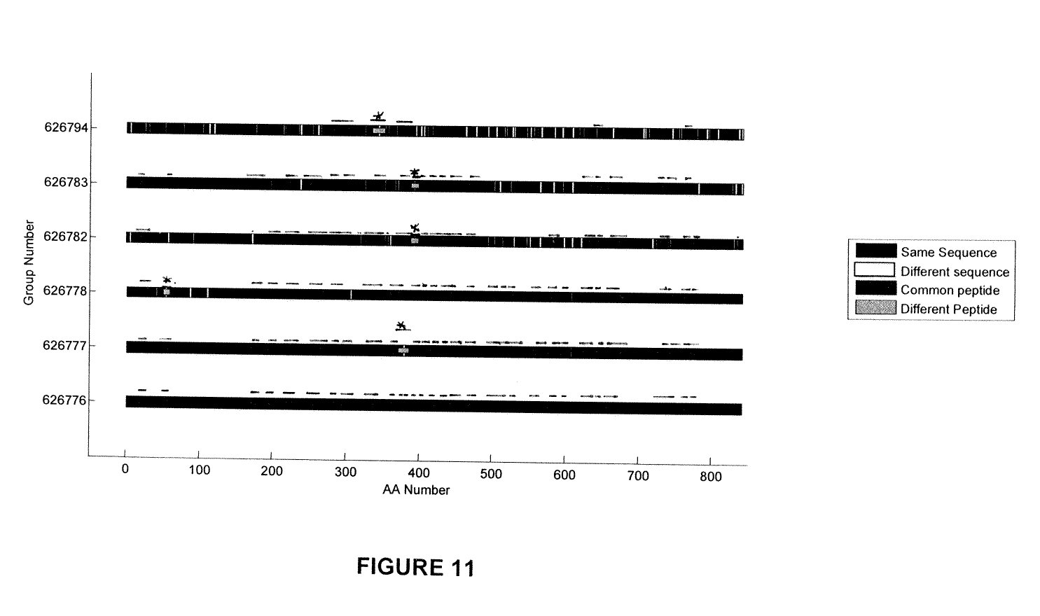

Each “island” in FIG. 10 appears to comprise closely related proteins which appear to be variants of the same protein. FIG. 11 shows the relation among groups in the first island using Group number 627667 as a reference. It is evident that: the proteins contain extensive regions with the same sequences (blue)•sequence difference were minor (yellow), most peptides are shared (red)•different groups were defined by a few peptides (green) corresponding to sequence difference.

This confirms that proteins in each island are highly related, probably as a results of sequence redundancy among species.

Final Results

Groups in each island were collapsed together and grouping repeated. Seven of the 8 most abundant proteins corresponded to those in the ABRF samples. 1 ABRF protein, horseradish peroxidase, was not identified since the search taxonomy was limited to mammals (table 3)

Relative Abundance

Relative abundance of 6/8 ABRF proteins was estimated from the ratio of spectral counts. Estimates were not possible for: Horseradish peroxidase since this was not identified; Beta Casein which was only identified in Sample I, where it was in the highest abundance.

These estimates corresponded to well to relative abundances provided by ABRF.

Conclusions

Seven of the eight proteins in ABRF sample were identified conclusively. Estimates of their relative abundances in the two samples based on spectral counts agreed well with expected values; Protein identification by data base search is complex if taxonomy is unrestricted.

### REFERENCES

[1] R. Aebersold and M. Mann, “Mass spectrometry-based proteomics,” Nature, vol. 422, pp. 198-207, 2003.
[2] D. N. Perkins, D. J. Pappin, D. M. Creasy, and J. S. Cottrell, “Probability-based protein identification by searching sequence databases using mass spectrometry data,” Electrophoresis, vol. 20, pp. 3551-67, 1999.
[3] J. Eng, A. McCormack, and J. R. I. Yates, “An approach to correlate tandem mass spectral data of peptides with amino acid sequences in protein data base,”J. Am. Soc. Mass Spectrom., vol. 5, pp. 976-989, 1994.
[4] F. Blondeau, B. Ritter, P. D. Allaire, S. Wasiak, M. Girard, N. K. Hussain, A. Angers, V. Legendre-Guillemin, L. Roy, D. Boismenu, R. E. Kearney, A. W. Bell, J. J. Bergeron, and P. S. McPherson, “Tandem MS analysis of brain clathrin-coated vesicles reveals their critical involvement in synaptic vesicle recycling,” Proc Natl Acad Sci USA, vol. 101, pp. 3833-8, 2004.
[5] H. Liu, R. G. Sadygov, and J. R. Yates, 3rd, “A model for random sampling and estimation of relative protein abundance in shotgun proteomics,” Anal Chem, vol. 76, pp. 4193-201, 2004.
[6] L. J. Foster, C. L. De Hoog, and M. Mann, “Unbiased quantitative proteomics of lipid rafts reveals high specificity for signaling factors,” Proc Natl Acad Sci USA, vol. 100, pp. 5813-8, 2003.
[7] A. I. Nesvizhskii, A. Keller, E. Kolker, and R. Aebersold, “A statistical model for identifying proteins by tandem mass spectrometry,” Anal Chem, vol. 75, pp. 4646-58, 2003.
[8] D. A. Benson, I. Karsch-Mizrachi, D. J. Lipman, J. Ostell, and D. L. Wheeler, “GenBank,” Nucleic Acids Res, vol. 33, pp. D34-8, 2005.
[9] Z. Bencsath-Makkai, A. Bell, J. Bergeron, D. Boismenu, M. Harrison, W. R. J. Funnell, C. Mounier, J. Paiement, L. Roy, and R. E. Kearney, “CellMapBase—An Information System Supporting High Throughput Proteomics for the Cell Map Project,” presented at Annual International Conference of the IEEE Engineering in Medicine and Biology Society, Cancun, Mexico, 2003.

All references cited herein are incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

TABLE I
RESULTS OF ELIMINATING REDUNDANT
IDENTIFICATIONS

| | Protein Hits | Protein, Groups |
|---|---|---|
| Total number | 1,449 | 824 |
| Number with no shared | 512 | 660 |
| peptide | | |
| Percentage with no | 35.2 | 80.1 |
| shared peptides | | |

TABLE II
Protein Groups identified for the two ABRF samples.
Prorated queries is the number of spectra associated with each group.
Protein Groups for the ABRF Sample

| | | | | | PERCENT | PRORATED |
|---|---|---|---|---|---|---|
| | CLUSTERID | REFERENCE | DESCRIPTION | SPECIES | COVERAGE | QUERIES |
| 1 | 626780 | Q362R2 | ALB protein | Bos taurus | 62.4 | 325.9 |
| 2 | 625784 | P80025 | Lactoperoxidase precursor LPO | Bos taurus | 45.6 | 206.0 |
| 3 | 626785 | 76365302 | hypothetical protein LOC531682 [Bos taurus] | Bos taurus | 49.1 | 119.0 |
| 4 | 626803 | 6P00751 | Trypsin precursor | pig | 46.4 | 116.0 |
| 5 | 626781 | 2P02769 | Serum albumin precursor | cow | 55.6 | 101.5 |
| 8 | 626787 | P00915 | Carbonic anhydrase 1 Carbonic anhydrase I Carbonat | Homo sapiens | 65.4 | 46.0 |
| 7 | 626812 | P11839 | Beta-casein precursor | Ovis aries | 15.8 | 14.0 |
| 8 | 626796 | 11P02768 | Serum albumin precursor | human | 7.4 | 7.3 |
| 9 | 626801 | Q6B32D | Serum albumin | Elephas maximus | 4.3 | 7.2 |
| 10 | 626805 | IP100717764.1 | SWISS-PROT:P30922 ENSEMBL:ENS8BTAP000000 [symbol] | Bos taurus | 17.4 | 5.0 |
| 11 | 626809 | 4P13645 | Keratin, type I cytoskeletal 10 | human | 8.8 | 3.0 |
| 12 | 626815 | 753 | seminal RNase (aa 47-124) [Bos taurus] | Bos taurus | 35.9 | 3.0 |
| 13 | 626819 | 3P04264 | Keratin, type II cytoskeletal 1 | human | 3.4 | 3.0 |
| 14 | 626826 | 1P33049 | Alpha-S2 casein precursor | goat | 4.9 | 3.0 |
| 15 | 626806 | Q3T101 | Hypothetical protein | Bos taurus | 14.5 | 2.0 |
| 16 | 626813 | Q8WVP4 | Quiescin Q6, isoform b | Homo sapiens | 4.1 | 2.0 |
| 17 | 626816 | UPI00001FE219 | thrombospondin 1 precursor | Homo sapiens | 2.1 | 2.0 |
| 18 | 626817 | 539969 | lysozyme homolog AT-2, bone - rat (fragments) | Rattus norvegicus | 100.0 | 2.0 |
| 19 | 626818 | IPI00718529.1 | TREMBL:Q2KJ32 ENSEMBL:ENSBTAP0000001064 [symbol] | Bos taurus | 6.8 | 2.0 |
| 20 | 626821 | Q9N273 | Kappa-casein | Bos indicus | 11.3 | 1.0 |
| 21 | 626822 | UPI0000112E69 | Carbonic Anhydrase II | Homo sapiens | 4.7 | 1.0 |
| 22 | 626824 | 1P10760 | Adenosythornocysteinase | Norway rat | 3.0 | 1.0 |
| 23 | 626825 | UPI00001104E7 | Angiogenin | Bos taurus | 7.2 | 1.0 |
| 24 | 626827 | 73970109 | PREDICTED: similar to 3-hydroxyanthranilate 3,4-dio [symbol] | Canis familiaris | 4.0 | 1.0 |
| 25 | 626828 | Q9N212 | Protein C inhibitor precursor Serine | Bos taurus | 6.2 | 1.0 |
| | | | (Or cysteine) prot [symbol] | | | |
| 26 | 626829 | 818028 | phosphorylase (aa 760-840) [Rattus norvegicus] | Rattus norvegicus | 11.3 | 1.0 |
| 27 | 626776 | 2P00489 | Glycogen phosphorylase, muscle form | rabbit | 52.8 | 0.0 |
| 28 | 626777 | UPI0000110764 | Glycogen Phosphorylase, Muscle Form | O. cuniculue | 52.9 | 0.0 |
| 29 | 628778 | 223003 | phosphorylase b, glycogen | O. cuniculue | 50.9 | 0.0 |
| 30 | 626779 | P02769 | Serum albumin precursor Allergen Bos d 6 BSA | Bos taurus | 65.9 | 0.0 |
| 31 | 626782 | NP_001009192.1 | muscle glycogen phosphorylase [Ovis aries] | unidentified | 38.4 | 0.0 |
| 32 | 626783 | UPI00004BCE81 | unknown | Canis familiaris | 37.3 | 0.0 |
| 33 | 626786 | P14639 | Serum albumin precursor | Ovis aries | 24.4 | 0.0 |
| 34 | 626788 | Q91X12 | Mutant catalase | Mus musculus | 21.3 | 0.0 |
| 35 | 626789 | NP_999466.1 | catalase [Sus scrofa] | unidentified | 19.3 | 0.0 |
| 36 | 626790 | 1P04040 | Catalase | human | 16.9 | 0.0 |
| 37 | 626791 | NP_001002964.1 | Catalase [Canis familiaris] | unidentified | 16.1 | 0.0 |
| 38 | 626792 | Q3UZE7 | 8 days embryo whole body cDNA, RIKEN full-length 4 | Mus musculus | 18.4 | 0.0 |
| 39 | 626793 | Q7YSG3 | Serum albumin precursor Allergen Fel d 2 | Felis catus | 9.2 | 0.0 |
| 40 | 626794 | 1P11216 | Glycogen phosphorylase, brain form | human | 9.8 | 0.0 |
| 41 | 626795 | P00661 | Ribonuclease pancreatic RNase 1 RNase A | Capra hircus | 76.6 | 0.0 |
| 42 | 626796 | P00656 | Ribonuclease pancreatic RNase 1 RNase A | Bison bison | 76.6 | 0.0 |
| 43 | 626797 | P07848 | Ribonuclease pancreatic RNase 1 RNase A | Gazella thomsonii | 76.6 | 0.0 |
| 44 | 626799 | 2P49822 | Serum albumin precursor | dog | 7.2 | 0.0 |
| 46 | 626800 | 73966878 | PREDICTED: similar to Lactoperoxidase precursor (LI | Canis familiaris | 8.4 | 0.0 |
| 46 | 626802 | Q6R461 | Lactoperoxidase | M. auratus | 6.1 | 0.0 |
| 47 | 626804 | 29P00556 | Ribonuclease pancreatic precursor | cow | 45.2 | 0.0 |
| 48 | 626806 | Q91WA0 | Lactoperoxidase | Mus musculus | 5.1 | 0.0 |
| 49 | 626807 | P22079 | Lactoperoxidase precursor LPO Salivary peroxidase Σ | Homo sapiens | 4.8 | 0.0 |
| 50 | 626810 | P07849 | Ribonuclease pancreatic RNase 1 RNase A | B. tragocamelus | 50.8 | 0.0 |
| 51 | 626811 | P00558 | Ribonuclease pancreatic RNase 1 RNase A | Tragelaphus oryx | 50.8 | 0.0 |
| 52 | 626814 | 2P07724 | Serum albumin precursor | house mouse | 6.5 | 0.0 |
| 53 | 626820 | 76713340 | PREDICTED: similar to immunoglobulin lambda-like p | Bos taurus | 18.5 | 0.0 |
| 54 | 626823 | 248147 | beta-casein A2 variant [cattle, Peptide Partial, 46 aa, | Bos taurus | 41.3 | 0.0 |
| 55 | 626830 | UPI00005070E3 | PREDICTED: similar to stabilin-2 | Rattus norvegicus | 0.5 | 0.0 |
| 58 | 626831 | 76615216 | PREDICTED: similar to Resin precursor, partial (Bos | Bos taurus | 0.5 | 0.0 |
| 57 | 626832 | P00762 | Anionic trypsin-1 precursor Anionic trypsin I Pretrypsi | Rattus norvegicus | 8.1 | 0.0 |
| 58 | 626833 | NP_032499.1 | keratin complex 2, basic, gene 1 [Mus musculus] | unidentified | 1.9 | 0.0 |
| 59 | 626834 | Q8BLW1 | Adult male aorta and vein cDNA, RIKEN full-length en | Mus musculus | 1.0 | 0.0 |

TABLE III
Protein groups and spectral counts after highly similar
groups are collapsed together. Proteins matching the ABRF
samples are indicated with an asterix
Final Protein List

| | | TOTAL | SAMPLE 1 | SAMPLE 2 | |
|---|---|---|---|---|---|
| CMBSEQID | DESCRIPTION | QUERIES | QUERIES | QUERIES | RATIO |
| [symbol] 21478 | Serum albumin precursor Allergen Bos d 6 BSA | 497 | 257 | 240 | 0.9 |
| [symbol] 69613 | Lactoperoxidase precursor LPO | 206 | 104 | 104 | 1.0 |
| [symbol] 258 | Glycogen phosphorylase, muscle form | 178 | 2 | 174 | 87.0 |
| [symbol] 3320409 | Catalase | 119 | 93 | 26 | 0.3 |
| 19160 | Trypsin precursor | 116 | 58 | 58 | 1.0 |
| [symbol] 87406 | Cartonic anhydrase | 46 | 17 | 29 | 1.7 |
| [symbol] 41849 | Ribonuclease pancreatic RNase 1 RNase A | 26 | 10 | 16 | 1.6 |
| [symbol] 196 | Beta-casein precursor | 12 | 12 | 0 | 0.0 |
| 3384430 | SWISS-PROT:P30922: similar to chitinase 3-like 1 isoform 2 | 5 | 2 | 3 | 1.5 |
| 69653 | Keratin, type I cytoskeletal 10 | 3 | 0 | 3 | |
| 10504 | Alpha-S2 casein precursor | 3 | 3 | 0 | |
| 3323085 | Hypothetical protein | 2 | 2 | 0 | |
| 3200175 | Hypothetical protein | 2 | 2 | 0 | |
| 130837 | lysozyme homolog AT-2, bone - rat (fragments) | 2 | 2 | 0 | |
| 90453 | Keratin, type II cytoskeletal 1 | 2 | 0 | 2 | |
| 16617 | thrombospondin 1 precursor | 2 | 0 | 2 | |
| 3465 | Quiescin Q6, isoform b | 2 | 0 | 2 | |
| 3280346 | PREDICTED: similar to 3-hydroxyanthranilate 3,4-dioxygenase (3-HAO) | 1 | 1 | 0 | |
| 148437 | Angiogenin | 1 | 0 | 1 | |
| 105809 | phosphorytase (aa 760-840) | 1 | 0 | 1 | |
| 54409 | S-Adenosythomocysteine Hydrolase | 1 | 1 | 0 | |
| 39122 | Protein C inhibitor precursor Serine (Or cysteine) proteinase inhibitor | 1 | 0 | 1 | |
| 19180 | Carbonic Anhydrase II | 1 | 0 | 1 | |
| 483 | Kappa-casein | 1 | 1 | 0 | |
| 3368768 | Similar to immunoglobulin lambda-like polypeptide 1 precursor | 0 | 0 | 0 | |

| | (Immunoglobulin-related 14.1 protein) | | | |
|---|---|---|---|---|
| 3361675 | TREMBL:Q6Q144 REFSEQ:XP_618382 PREDICTED: similar to Reefin | 0 | 0 | 0 |
| | precursor, partial | | | |
| 2734641 | PREDICTED: similar to stabilin-2 | 0 | 0 | 0 |
| 242220 | Adult male aorta and vein cDNA, RIKEN full-length enriched library | 0 | 0 | 0 |
| 79094 | beta-casein A2 variant [ | 0 | 0 | 0 |
| 75054 | Anionic trypsin-1 precursor Anionic trypsin I Pretrypsinogen I | 0 | 0 | 0 |
| 37492 | keratin complex 2, basic, gene 1 [Mus musculus] | 0 | 0 | 0 |

1. A method for identifying one or more proteins in a mixture of proteins said method comprising:
a) providing peptides derived from said mixture of proteins;
b) obtaining mass spectra of said peptides to identify said peptides by comparing said mass spectra with spectra of a standardized database;
c) matching said identified peptides with proteins in a database to generate a protein hits (PHs) list, each of said PHs having an associated peptides set; and
d) identifying PHs having an associated peptides set that is included in at least one other PH-associated peptide set; and
e) removing said identified PHs from said list and wherein remaining PHs provides an identification of said one or more proteins.

2. The method as claimed in claim 1 further comprising grouping said identified PHs that share a same set of peptides in primary protein groups and wherein each of said primary protein group identifies a non-redundant PH.

3. The method as claimed in claim 2 further comprising:
a) combining all primary protein groups that share at least one common characteristic among said non-redundant PH to generate secondary protein groups and
b) identifying a non-redundant PH for each of said secondary protein groups based on said characteristic.

4. The method as claimed in claim 3 wherein said characteristic is sharing at least one common peptide among said non-redundant PH of said primary protein groups.

5. The method as claimed in claim 4 further comprising:
a) assigning a connectivity value to each of said primary protein group wherein said connectivity value is related to the number of primary protein groups with which a given primary protein group shares at least one peptide and wherein said identifying is based on said connectivity.

6. The method as claimed in any one of claims 1-5 further comprising a step of providing relative abundance of a PH.

7. The method as claimed in claim 6 wherein said relative abundance is the number of peptides associated with all PHs in a primary or secondary protein group.

8. The method as claimed in claim 7 wherein said relative abundance is a sum of peptides unique to said primary or secondary protein group and peptides that are shared with other protein groups and wherein said number of shared peptides is weighted as a function of unique peptides.

9. A computer-readable medium comprising instructions for causing a computer linked to one or several mass spectrometers and to one or more biological sequence databases to perform the steps of the method of any one of claims 1-8.

10. A system comprising a computer linked to one or more mass spectrometers and to one or more biological sequence databases, said computer comprising a program for performing the steps of the method of any one of claims 1-8.

11. A method for reducing redundancy in a protein hits list, comprising:
a) associating a set of peptides with each protein of said protein hits to generate PHs-associated peptide sets;
b) comparing said set PHs-associated peptide sets;
c) identifying PHs having an associated peptides set that is included in at least one other PH-associated peptides set; and
d) removing said identified PHs from said list and wherein remaining PHs provides an identification of said one or more proteins.

12. The method as claimed in claim 11 further comprising grouping said identified PHs that share a same set of peptides in primary protein groups and wherein each of said primary protein group identifies one non-redundant PH.

13. The method as claimed in claim 12 further comprising:
a) combining all primary protein groups that share at least one common characteristic among said non-redundant PH to generate secondary protein groups and
b) identifying a non-redundant PH for each of said secondary protein groups based on said characteristic.

14. The method as claimed in claim 13 wherein said characteristic is sharing at least one common peptide among said non-redundant PH of said primary protein groups.

15. The method as claimed in claim 14 further comprising:
a) assigning a connectivity value to each of said primary protein group wherein said connectivity value is related to the number of primary protein groups with which a given primary protein group shares at least one peptide and wherein said identifying is based on said connectivity.

16. The method as claimed in any one of claims 11-15 further comprising a step of providing relative abundance of a PH.

17. The method as claimed in claim 16 wherein said relative abundance is the number of peptides associated with all PHs in a primary or secondary protein group.

18. The method as claimed in claim 17 wherein said relative abundance is a sum of peptides unique to said primary or secondary protein group and peptides that are shared with other protein groups and wherein said number of shared peptides is weighted as a function of unique peptides.

19. A computer-readable medium comprising instructions for causing a computer linked to one or several mass spectrometers and to one or more biological sequence databases to perform the steps of the method of any one of claims 11-18.

20. A system comprising a computer linked to one or more mass spectrometers and to one or more biological sequence databases, said computer comprising a program for performing the steps of the method of any one of claims 11-18.
